(19) United States
(12) Patent Application Publication
(10) Pub. No.: US 2026/0269009 A1
(43) Pub. Date: Sep. 10, 2026

(54) USING ADAPTIVE SEQUENCING AND HARDWARE-ACCELERATED STORAGE TO ACCELERATE METAGENOMIC SAMPLE ANALYSIS

(71) Applicant: University of Washington, Seattle, WA (US)

(72) Inventors: Russell N. Van Gelder, Seattle, WA (US); Kenji Nakamichi, Seattle, WA (US); Aaron Lee, Seattle, WA (US)

(73) Assignee: University of Washington, Seattle, WA (US)

(21) Appl. No.: 18/876,365

(22) PCT Filed: Jun. 21, 2023
(86) PCT No.: PCT/US2023/068840

§ 371 Date: Dec. 18, 2024

Related U.S. Application Data
(60) Provisional application No. 63/355,054, filed on Jun. 23, 2022.

(51) Int. Cl. G16B 30/00; G16B 20/20; G16B 45/00

(52) U.S. Cl. CPC G16B 30/00; G16B 20/20; G16B 45/00

(57) ABSTRACT

In some embodiments, a computer-implemented method of predicting a molecule type using segment read information is provided. A computing device receives a segment read for a molecule. The computing device determines one or more k-mers for the segment read. For each k-mer of the one or more k-mers, the computing device retrieves one or more records from a hash table using the k-mer as a key, wherein each record includes an identifier associated with a molecule type. The computing device generates a molecule type prediction for the segment read based on the identifiers of the retrieved one or more records.

### CROSS-REFERENCE TO RELATED APPLICATION

This application claims the benefit of Provisional Application No. 63/355,054, filed Jun. 23, 2022, the entire disclosure of which is hereby incorporated by reference herein for all purposes.

### BACKGROUND

Metagenomic sequencing refers to process of identifying one or more microbes, viruses, or other organisms from a complex mix via bioinformatics. This technique has been shown to be capable of identifying potential pathogens without a priori knowledge of differential diagnosis, and may theoretically be applied to any DNA- or RNA-based lifeform. However, current pipelines such as Kraken, Dragen, or similar pipelines are typically performed on DNA samples after full sequencing has been accomplished which, depending on platform, can induce delays of 24-48 hours before potential identification.

The SMART metagenomic pipeline was designed to perform very rapid sequence identification using hashtable lookup methods. However, the SMART metagenomic pipeline used a high-performance computing cluster, which is not readily available in most processing environments. What is desired are techniques for performing metagenomic analysis that can accelerate the analysis on commodity-level computing hardware. What is also desired are techniques that can help accelerate the generation of data by leveraging adaptive sequencing functionality available in modern nanopore sequencing technology.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In some embodiments, a computer-implemented method of predicting a molecule type using segment read information is provided. A computing device receives a segment read for a molecule. The computing device determines one or more k-mers for the segment read. For each k-mer of the one or more k-mers, the computing device retrieves one or more records from a hash table using the k-mer as a key, wherein each record includes an identifier associated with a molecule type. The computing device generates a molecule type prediction for the segment read based on the identifiers of the retrieved one or more records.

In some embodiments, a non-transitory computer-readable medium having computer-executable instructions stored thereon is provided. The instructions, in response to execution by one or more processors of a computing device, cause the computing device to perform actions of a method as described above.

In some embodiments, a computing device configured to perform a method as described above is provided.

In some embodiments, a system for predicting molecule types is provided. The system comprises a sequencing device and a computing device communicatively coupled to the sequencing device. The computing device is configured to perform a method as described above.

In some embodiments, a method of predicting a molecule type in a sample is provided. The sample is obtained from a subject, and is applied to a sequencing device configured to generate sequence read information. The sequencing device transmits the sequence read information to a computing device. The computing device performs actions of a method as described above to predict the molecule type.

### BRIEF DESCRIPTION OF THE DRAWINGS

The foregoing aspects and many of the attendant advantages of this disclosure will become more readily appreciated as the same become better understood by reference to the following detailed description, when taken in conjunction with the accompanying drawings, wherein:

FIG. 1 is a schematic illustration of a system for nanopore-based analysis according to various aspects of the present disclosure.

FIG. 2 is a schematic illustration of a non-limiting example embodiment of a flow cell according to various aspects of the present disclosure.

FIG. 3 is a block diagram that illustrates aspects of a non-limiting example embodiment of a metagenomic computing system according to various aspects of the present disclosure.

FIG. 4A-FIG. 4B are a flowchart that illustrates a non-limiting example embodiment of a method of predicting a molecule type according to various aspects of the present disclosure.

### DETAILED DESCRIPTION

In some embodiments of the present disclosure, three technologies are combined—nanopore sequencing, adaptive sequencing (in which sequencing is aborted for specific DNA molecules, in our case human sequence), and hardware-accelerated hash table storage—to provide techniques that can drastically accelerate metagenomic analysis of samples. While a variety of applications for such accelerated analysis are possible, in some embodiments, the techniques disclosed herein may be used to perform real-time pathogen discovery while sequence data is being generated.

In some embodiments, the SMART metagenomic pipeline has been updated to allow a hash table storing molecule type information to fit in a memory of a graphical processing unit (GPU) or other readily available commodity-level specialized processor for conducting highly parallelized computations. In some embodiments of the present disclosure, k-mers (e.g., 30-mers) based on segment reads are used as keys to a hash table (e.g., a UN64 value). Each record in the hash table stores at least an identifier (e.g., a UN32 value) that represents a molecule type associated with the k-mer. In some embodiments, the identifier may link to a separate reference data store that provides text (or other values) that identifies the associated molecule type. By compressing the k-mer and molecule type information in the records in the hash table to a UN64 value and a UN32 value, a size of the hash table previously used in SMART can be reduced from 4 TB (in the previous sharded implementation of SMART) to 72 GB, thus fitting within the memory of a single commodity-level graphical processing unit. This allows the entire hash table to be queried at once, instead of requiring multiple queries to multiple shards as in the previous SMART implementation.

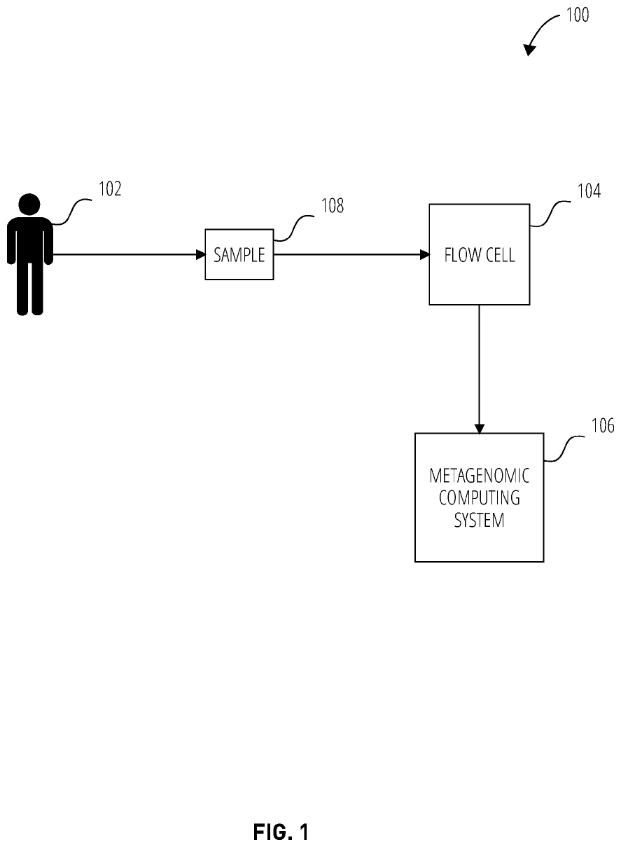

FIG. 1 is a schematic illustration of a system for nanopore-based analysis according to various aspects of the present disclosure. As shown, in the system 100, a sample 108 is obtained from a subject 102 using known techniques. The sample 108 may be a tissue biopsy, a swab, a blood sample, or any other suitable type of sample 108. The sample 108 is prepared (e.g., combined with one or more buffers, enzymes, etc.), and the prepared sample 108 is provided to a flow cell 104 of a sequencing device. Some non-limiting examples of a sequencing device are a MinION sequencing device, a GridION sequencing device, and a PromethION sequencing device, all of which are provided by Oxford Nanopore Technologies plc. Some non-limiting examples of devices for implementing a flow cell 104 are a Flongle Flow Cell, a MinION Flow Cell, and the PromethION Flow Cell, each also provided by Oxford Nanopore Technologies plc. The flow cell 104 generates signals based on interactions between the sample 108 and the nanopores of the flow cell 104, and provides the signals to the metagenomic computing system 106 for analysis.

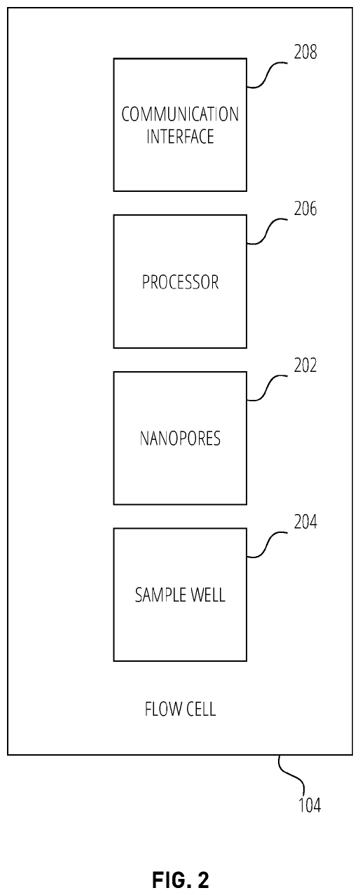

FIG. 2 is a schematic illustration of a non-limiting example embodiment of a flow cell according to various aspects of the present disclosure. As shown, the flow cell 104 includes a sample well 204, a plurality of nanopores 202, a processor 206, and a communication interface 208. The sample well 204 is configured to accept the sample 108 (e.g., to receive drops of sample 108 from a pipette) and to provide the sample 108 to the plurality of nanopores 202. The processor 206 is configured to control a voltage applied to the plurality of nanopores 202 and to read signals generated by the nanopores 202. In some embodiments, the processor 206 may be configured to receive commands via the communication interface 208 to abort sequencing in a given nanopore of the plurality of nanopores 202, in which case a voltage is applied to the given nanopore that causes the molecule to be ejected from the given nanopore so that a different molecule may be processed by the given nanopore.

In some embodiments, the processor 206 may also be configured to segment the signals generated by the nanopores 202 into a plurality of segmented events, each segmented event representing an interaction of a molecule with a nanopore 202 of the plurality of nanopores 202. In some embodiments, the processor 206 may be further configured to perform basecalling (determining identities of one or more amino acids interacting with the nanopore as represented by one or more segmented events).

In some embodiments, the communication interface 208 is configured to transmit the signals detected by the processor 206, the segmented events, and/or the basecalling results to another device, such as the metagenomic computing system 106, using a wired or wireless network, a USB connection, or any other suitable communication technique. In some embodiments, the processor 206, communication interface 208, and potentially other components (such as a computer-readable medium) may be implemented on an ASIC or FPGA that is part of the flow cell 104.

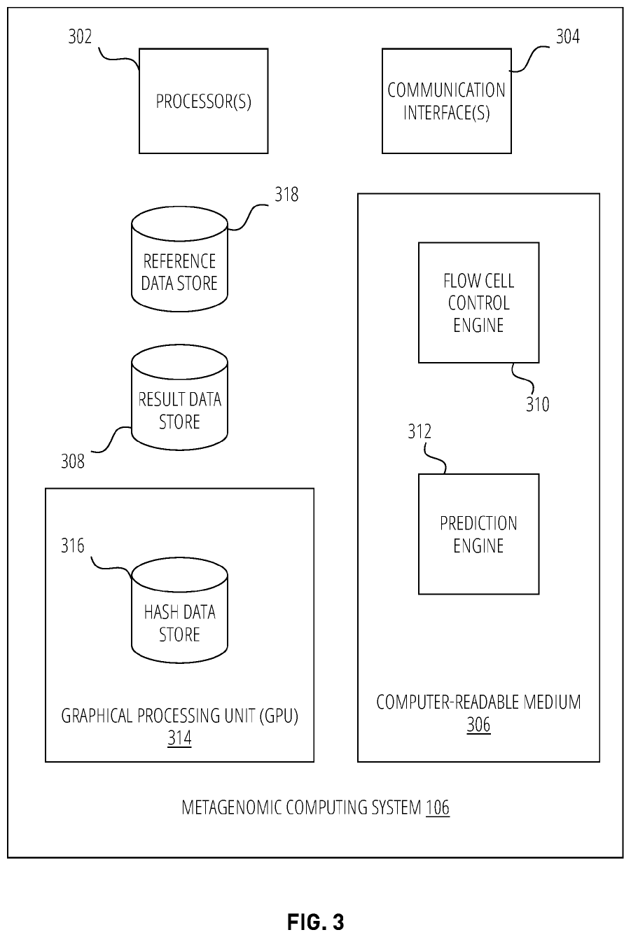

FIG. 3 is a block diagram that illustrates aspects of a non-limiting example embodiment of a metagenomic computing system according to various aspects of the present disclosure. The illustrated metagenomic computing system 106 may be implemented by any computing device or collection of computing devices that includes the illustrated features, including but not limited to a desktop computing device, a laptop computing device, a mobile computing device, a server computing device, a computing device of a cloud computing system, and/or combinations thereof. The metagenomic computing system 106 is configured to process basecalling information to efficiently determine predictions of molecule types, which can then be used for any suitable purpose, including but not limited to identifying potential pathogens indicated by the sample 108, detecting phenotypes, detecting a genetic alteration associated with a specific cancer, detecting a point mutation, or any other suitable purpose.

As shown, the metagenomic computing system 106 includes one or more processors 302, one or more communication interfaces 304, a result data store 308, a reference data store 318, a result data store 308, a graphical processing unit 314, and a computer-readable medium 306.

In some embodiments, the processors 302 may include any suitable type of general-purpose computer processor. In some embodiments, the processors 302 may include one or more special-purpose computer processors or AI accelerators optimized for specific computing tasks, including but not limited to graphical processing units (GPUs), vision processing units (VPTs), and tensor processing units (TPUs).

In some embodiments, the illustrated graphical processing unit 314 may be one of the processors 302, or may be separate from the processors 302. In some embodiments, the graphical processing unit 314 is a special-purpose computer processor that includes a plurality of processing cores and a memory that are optimized for efficient parallel computation. In some embodiments, the graphical processing unit 314 may be a commercially available product, such as a GEFORCE® RTX 4090, from NVIDIA®, which has 24 GB of GPU memory and 16,384 CUDA processing cores; a RADEON™ RX 7900 XTX, which has 24 GB of GPU memory and 96 compute units; or any other type of commercially available graphical processing unit 314. The graphical processing unit 314 hosts a hash data store 316, which, as described above, stores a hash table that includes records that associate a key (e.g., a unique k-mer value) with one or more entries that at least include identifiers (e.g., UN32 values) that link to further information about an associated molecule type in the reference data store 318. In some embodiments, the entire hash data store 316 fits within the GPU memory of the graphical processing unit 314. Any suitable engine may be used to manage the hash data store 316 within the graphical processing unit 314. In some embodiments, the WarpCore engine described in Junger et al., WarpCore: A Library for fast Hash Tables on GPUs, 2020 IEEE 27th International Conference on High Performance Computing, Data, and Analytics (HiPC), the entire disclosure of which is hereby incorporated by reference herein for all purposes, and available as an open source project, may be used to take advantage of parallelism in the graphical processing unit 314 to provide the hash data store 316 in a highly efficient and performant manner.

In some embodiments, the communication interfaces 304 include one or more hardware and or software interfaces suitable for providing communication links between components. The communication interfaces 304 may support one or more wired communication technologies (including but not limited to Ethernet, FireWire, and USB), one or more wireless communication technologies (including but not limited to Wi-Fi, WiMAX, Bluetooth, 2G, 3G, 4G, 5G, and LTE), and/or combinations thereof.

As shown, the computer-readable medium 306 has stored thereon logic that, in response to execution by the one or more processors 302, cause the metagenomic computing system 106 to provide a flow cell control engine 310, and a prediction engine 312.

As used herein, “computer-readable medium” refers to a removable or nonremovable device that implements any technology capable of storing information in a volatile or non-volatile manner to be read by a processor of a computing device, including but not limited to: a hard drive; a flash memory; a solid state drive; random-access memory (RAM); read-only memory (ROM); a CD-ROM, a DVD, or other disk storage; a magnetic cassette; a magnetic tape; and a magnetic disk storage.

In some embodiments, the flow cell control engine 310 is configured to transmit commands to the flow cell 104 in response to predictions made by the prediction engine 312, such as abort commands transmitted in response to a prediction that a molecule within a given nanopore 202 is of an undesired type. In some embodiments, the prediction engine 312 is configured to make predictions of molecule types by querying the hash data store 316 using keys based on k-mers of the basecalling information, and to store the predictions of the molecule types in the result data store 308.

Further description of the configuration of each of these components is provided below.

As used herein, “engine” refers to logic embodied in hardware or software instructions, which can be written in one or more programming languages, including but not limited to C, C++, C#, COBOL, JAVA™, PHP, Perl, HTML, CSS, JavaScript, VBScript, ASPX, Go, and Python. An engine may be compiled into executable programs or written in interpreted programming languages. Software engines may be callable from other engines or from themselves. Generally, the engines described herein refer to logical modules that can be merged with other engines, or can be divided into sub-engines. The engines can be implemented by logic stored in any type of computer-readable medium or computer storage device and be stored on and executed by one or more general purpose computers, thus creating a special purpose computer configured to provide the engine or the functionality thereof. The engines can be implemented by logic programmed into an application-specific integrated circuit (ASIC), a field-programmable gate array (FPGA), or another hardware device.

As used herein, “data store” refers to any suitable device configured to store data for access by a computing device. One example of a data store is a highly reliable, high-speed relational database management system (DBMS) executing on one or more computing devices and accessible over a high-speed network. Another example of a data store is a key-value store. However, any other suitable storage technique and/or device capable of quickly and reliably providing the stored data in response to queries may be used, and the computing device may be accessible locally instead of over a network, or may be provided as a cloud-based service. A data store may also include data stored in an organized manner on a computer-readable storage medium, such as a hard disk drive, a flash memory, RAM, ROM, or any other type of computer-readable storage medium. One of ordinary skill in the art will recognize that separate data stores described herein may be combined into a single data store, and/or a single data store described herein may be separated into multiple data stores, without departing from the scope of the present disclosure.

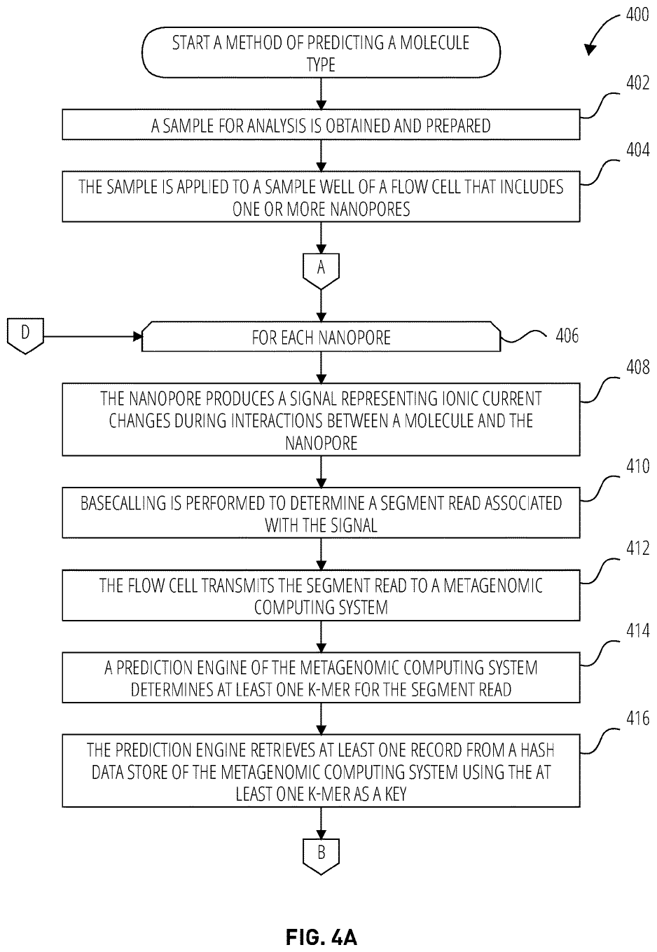

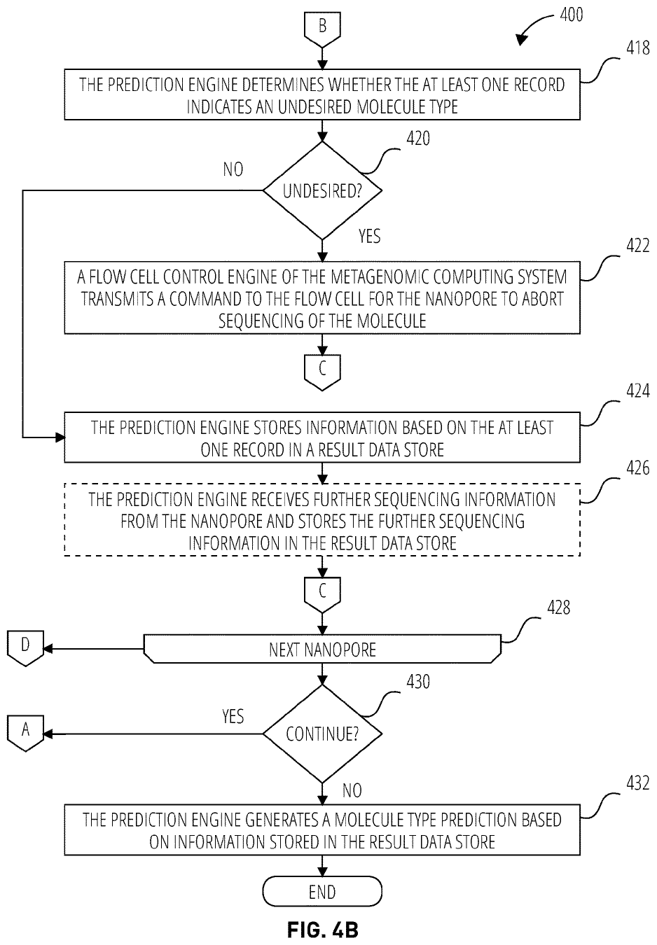

FIG. 4A-FIG. 4B are a flowchart that illustrates a non-limiting example embodiment of a method of predicting a molecule type according to various aspects of the present disclosure. In the method 400, various techniques are used to accelerate the computation of the prediction of the molecule type, including using abort functionality of the flow cell 104, and using a hardware-accelerated hash data store 316 for queries related to the sequence information generated by the flow cell 104. The molecule type predicted by the method 400 may be any category of molecule that can be predicted based on a k-mer. Some non-limiting examples of molecule types that may be predicted by the method 400 include, but are not limited to, a cell type, a protein family, a virus type, a tissue type, a species of an organism, an insertion, a deletion, a point mutation, presence or absence of a gene, and a genetic alteration associated with a specific cancer.

From a start block, the method 400 proceeds to block 402, where a sample 108 for analysis is obtained and prepared. At block 404, the sample 108 is applied to a sample well 204 of a flow cell 104 that includes one or more nanopores 202. The actions of block 402 and block 404 are typical for nanopore analysis of samples 108 and are known to those of ordinary skill in the art. Such actions are further described in commercially available instructions for operation of commercially available flow cells 104, and so are not described in further detail herein for the sake of brevity.

The method 400 then advances through a continuation terminal (“terminal A”) to a for-loop defined between for-loop start block 406 and for-loop end block 428 wherein signals from each nanopore 202 of the one or more nanopores 202 are processed. While the method 400 illustrates the processing of the one or more nanopores 202 serially in the for-loop, one will recognize that in some embodiments, processing for two or more nanopores 202 may occur at least partially concurrently/in parallel, and processing of multiple molecules within a given nanopore 202 (i.e., more than one pass through the for-loop for the given nanopore 202) may occur within the method 400.

From the for-loop start block 406, the method 400 advances to block 408, where the nanopore 202 produces a signal representing ionic current changes during interactions between a molecule and the nanopore 202. At block 410, basecalling is performed to determine a segment read associated with the signal. The segment read includes a sequence of bases predicted by the flow cell 104 to be present in the molecule transiting the nanopore 202. At block 412, the flow cell 104 transmits the segment read to a metagenomic computing system 106. In some embodiments, instead of the flow cell 104 performing basecalling to determine the sequence of bases for the segment read, the flow cell 104 may transmit the signal representing the ionic current changes to the metagenomic computing system 106, and the metagenomic computing system 106 (e.g., the prediction engine 312 or another component) may perform the basecalling to determine the segment read. In some embodiments, instead of transmitting a complete segment read, the flow cell 104 may stream the called sequence of bases to the metagenomic computing system 106 as they become available.

At block 414, a prediction engine 312 of the metagenomic computing system 106 determines at least one k-mer for the segment read. The k-mer is a set of k bases from the segment read. In some embodiments, the k-mer may be the first k bases from the segment read. In some embodiments, the prediction engine 312 may use a sliding window to select k bases from the segment read to generate multiple k-mers (e.g., selecting bases 1 through k as a first k-mer, bases 2 through k+1 as a second k-mer, bases 3 through k+2 as a second k-mer, etc.).

Any suitable value for k may be used. One non-limiting example of a suitable value for k may be selected from a range of 25 to 35, such as 30. The value selected for k is a balance of how much sequencing is to be performed and how large the hash data store 316 will be to store entries for all potential k-mers. For example, in some embodiments of the hash data store 316, the size of the storage used for the hash data store 316 is 4 k, and so smaller k-mers may be desirable to reduce the size of the hash data store 316. If the method 400 is being used to analyze a limited genomic space, smaller values for k may be appropriate. For example, in a non-limiting example embodiment of the method 400 that was used to distinguish between human and non-human molecule types, a value of 25 for k was found to be appropriate. As another example, in a non-limiting example embodiment of the method 400 that is being used to detect variations within a human genome, a larger value for k such as 1024 may be more appropriate due to the greater similarity between molecule types.

At block 416, the prediction engine 312 retrieves at least one record from a hash data store 316 of the metagenomic computing system 106 using the at least one k-mer as a key. In some embodiments, one record may be retrieved for each k-mer. In some embodiments, multiple records may be retrieved for a given k-mer. Each k-mer may be associated with a unique molecule type, an ambiguous molecule type (e.g., more than one molecule type), or no known molecule type. In some embodiments, each record retrieved from the hash data store 316 may indicate whether the k-mer is associated with a unique molecule type, an ambiguous molecule type, or no known molecule type. In some embodiments, records that are retrieved that are not associated with a unique molecule type may be ignored, since the predictive strength of records that are not associated with a unique molecule type is lower.

In some embodiments, the records retrieved from the hash data store 316 may also be associated with additional information. For example, in some embodiments, the additional information may include a name of the molecule type associated with the k-mer. As another example, in some embodiments, the records from the hash data store 316 may be associated with an indication of a specific feature associated with the sequence represented by the k-mer, such as a chromosomal location, a specific mutation in a genetic locus, and/or a chemical modification (e.g., methylation, acetylation, or another chemical adduct). As another example, in some embodiments, additional information may be made available by the flow cell 104, such as whether specific bases are modified or non-modified. In some embodiments, instead of including the additional information, the record retrieved from the hash data store 316 may include a link to the additional information to be retrieved from the reference data store 318.

The method 400 then advances to a continuation terminal (“terminal B”). From terminal B (FIG. 4B), the method 400 proceeds to block 418, where the prediction engine 312 determines whether the at least one record indicates an undesired molecule type. In some embodiments, desired molecule types may be associated with an organism of interest, while undesired molecule types may be associated with another organism. For example, if the method 400 is attempting to search for molecule types associated with a pathogen such as an adenovirus, molecule types that are uniquely associated with the adenovirus (e.g., k-mers that are present within the adenovirus genome but not a human genome) may be desired molecule types, and molecule types that are not uniquely associated with the adenovirus (e.g., k-mers that are present within the human genome but not the adenovirus genome, or are present in both genomes) may be undesired molecule types.

The method 400 then advances to a decision block 420 that is based on the determination of whether the at least one record indicates an undesired molecule type. If the determination indicated that the at least one record does indicate an undesired molecule type, then the result of decision block 420 is YES, and the method 400 proceeds to block 422, where a flow cell control engine 310 of the metagenomic computing system 106 transmits a command to the flow cell 104 for the nanopore 202 to abort sequencing of the molecule. The command causes the flow cell 104 to apply a voltage that causes the molecule to be ejected from the nanopore 202, thus ending the sequencing of the molecule and allowing a different molecule to enter the nanopore 202 for subsequent processing. By using the abort command, the method 400 can avoid sequencing undesired molecule types, and thereby reduce the amount of processing time used to generate results. The method 400 then proceeds to a continuation terminal (“terminal C”) to jump to the end of the for-loop.

Returning to decision block 420 if the determination indicated that the at least one record does not indicate an undesired molecule type, then the result of decision block 420 is NO, and the method 400 proceeds to block 424. At block 424, the prediction engine 312 stores information based on the at least one record in a result data store 308 of the metagenomic computing system 106. In some embodiments, the prediction engine 312 may store information indicating that an instance of the molecule type indicated by the at least one record was found to be present in the sample 108, or indicating that the k-mer was found to be present in the sample 108.

At optional block 426, the prediction engine 312 receives further sequencing information from the nanopore 202 and stores the further sequencing information in the result data store 308. The actions of optional block 426 are illustrated and described as optional because, in some embodiments, determining the sequence beyond the k-mer is unimportant, as the mere presence of a k-mer uniquely associated with a desired molecule type is adequate information to be gathered from the molecule. In such embodiments, instead of gathering further sequencing information, the prediction engine 312 may transmit the abort command to the flow cell 104 because adequate information has already been gathered from the molecule. In other embodiments, the decision of whether the molecule represents a desired molecule type or undesired molecule type indicates whether it is worth gathering as much sequencing information from the molecule as possible, and so the actions of optional block 426 would gather the desired information.

The method 400 then continues through terminal C to a for-loop end block 428. At the for-loop end block 428, if more nanopores 202 remain to be processed (or subsequent signals are obtained from the same nanopore 202 for a different molecule to be processed), then the method 400 proceeds to a continuation terminal (“terminal D”) to return to the for-loop start block 406 and to process signals from the next nanopore 202. Otherwise, if no more nanopores 202 remain to be processed, then the method 400 proceeds to decision block 430.

At decision block 430, a determination is made regarding whether the method 400 should continue to process further signals from the nanopores 202. Any suitable determination may be used regarding whether the method 400 should continue. In some embodiments, the determination may be made based on whether a predetermined amount of time has elapsed, or a predetermined number of segment reads from the flow cell 104 have been processed. In some embodiments, the determination may be made based on whether a predetermined minimum number of records have been stored for a desired molecule type. In some embodiments, the determination may be made based on whether any of the nanopores 202 are still active.

If it is determined that the method 400 should continue, then the result of decision block 430 is YES, and the method 400 returns to terminal A to process more signals from the nanopores 202. In some embodiments, some or all of the nanopores 202 previously processed may again be processed on the next iteration. In some embodiments, a different set of nanopores 202 may be processed in the next iteration. Further, in some embodiments, settings for the method 400 may be adjusted. For example, the set of desired molecule types and undesired molecule types may be adjusted for the next iteration of the for-loop. One example of such processing would be if a sample 108 was being processed to search for multiple different pathogens. On a first iteration through the for-loop, any molecule types uniquely associated with a human may be ignored, and records for other molecule types may be stored. Once at least a threshold number of records for a given pathogen have been stored, then molecule types associated with the given pathogen may be added to the list of undesired molecule types for subsequent iterations in order to increase the amount of information gathered for other pathogens. In other words, after ignoring human molecule types and detecting a threshold number (e.g., 10,000) records for molecule types uniquely associated with strep pneumonia, then it can be assumed that strep pneumonia is present in the sample 108, and the molecule types for strep pneumonia can be added to the undesired molecule types in order to more efficiently collect data for other molecule types. Another situation in which this technique may be desirable would be in embodiments in which gene sequencing is being performed-once 10× coverage of a given gene is obtained, molecule types uniquely associated with the given gene may be added to the list of undesired molecule types because adequate coverage of the given gene had already been obtained.

If it is determined at decision block 430 that the method 400 should not continue, then the result of decision block 430 is NO, and the method 400 proceeds to block 432. At block 432, the prediction engine 312 generates a molecule type prediction based on information stored in the result data store 308. In some embodiments, the prediction engine 312 may conduct a statistical analysis to determine probabilities of one or more molecule types based on the records stored in the result data store 308, and may provide one or more of the most probable molecule types present in the sample 108 as the prediction of the molecule type. For example, the prediction engine 312 may determine a molecule type indicated most often by the records stored in the result data store 308 as the prediction of the molecule type. As another example, the prediction engine 312 may determine probabilities for multiple molecule types based on the relative percentage of the records stored in the result data store 308 that indicate the various molecule types. The prediction engine 312 may present the prediction of the molecule type on a display device of the metagenomic computing system 106, may transmit the prediction of the molecule type to another system, or may store the prediction of the molecule type for later use by the metagenomic computing system 106 or another system.

The method 400 then proceeds to an end block and terminates.

While the discussion above includes references to nanopore sequencing, in some embodiments, different sequencing techniques may be used. If these different sequencing techniques also support the abort command described above, then the method 400 may be used with these different sequencing technologies with little alteration. If these different sequencing techniques do not support the abort command, then the steps of the method 400 relating to the abort command may be ignored, but the technical benefits of using the hash table storage as described above may still be obtained.

### Description of Testing of Non-Limiting Example Embodiment

To test a non-limiting example embodiment of the present disclosure, a HIPAA-compliant study was designed, approved by Institutional Board Review, and conducted in accord with the Declaration of Helsinki. The BayNovation clinical trial was registered at clinicaltrials.gov (NCT01877694). Informed consent was obtained from all study subjects. Subjects were included if they were older than 18 years of age; had one of the following: recent upper respiratory tract infection, contact with an infected person, and/or had a recent visit to an eye care provider; at least 2 of 9 clinical signs indicative of conjunctivitis; onset less than 3 days prior to enrollment; and had a positive point-of-service AdV antigen screening test (Adeno Plus, Rapid Pathogen Screening, Inc., Sarasota, FL). Study subjects were recruited from centers in Brazil, Sri Lanka, and India. Conjunctival fornices were swabbed with polyester sample swabs and placed in sterile balanced salt solution prior to processing.

DNA was extracted from swabs using QIAgen QIAsymphony kit following manufacturer's instructions. Library preparation was completed by rapid PCR barcoding (SQK-RPB004) per the manufacturer's protocol, and two samples multiplexed per flow-cell (R9.4.1) in each experiment. Each sequencing experiment was run on the Oxford Nanopore MinION Mk1B device for 48 hrs, or until all the nanopores 202 were inactive.

In further detail, for each reaction, 3 ul of high molecular weight genomic DNA (~5 ng total) was incubated with 1 ul Fragmentation Mix (FRM) at 30° C. for 1 minute and 80° C. for 1 minute. Each sample was diluted with 20 ul nuclease-free water, assigned a unique barcode and 1 ul was added to the tagmented DNA. 25 ul of LongAmp Taq 2× Master Mix (NEB M0287) was added. Cycling conditions were an initial 3 minute denaturation at 95° C., followed by 14 cycles of 15-second denaturation at 95° C., 15 second annealing at 56° C., and 6 minute extension at 65° C., then an additional extension step for 6 minutes at 65° C. The barcoded DNA was bound to AMPure XP beads, pelleted on a magnet and washed twice with 200 ul 80% EtOH. The residual EtOH was removed, 11 ul nuclease-free water was added and after incubation for 3 minutes at RT, the AMPure XP beads were pelleted on a magnet. The eluate was then removed and 1 ul quantified by Qubit dsDNA HS assay. Two samples were multiplexed in each sequencing experiment by pooling the barcoded DNA in the appropriate ratios to obtain 50 fmol of DNA in 10 ul nuclease-free water. 1 ul rapid adapter mix (RAP) was added and the pool was incubated again for 5 minutes at RT. An R9.4.1 flow cell was brought to RT and upon passing QC, 30 ul of flush tether (FLT) was added to a new tube of flush buffer (FB) and 800 ul of this mixture was added to the flow cell via the priming port and incubated for at least 5 minutes. The loading mix was prepared with 34 ul of sequencing buffer (SB), 25.5 ul of loading beads (LB), 4.5 ul nuclease-free water, and 11 ul of the barcoded DNA pool. The priming step was completed by adding an additional 200 ul of the flush buffer mixture with the sample port (i.e., the sample well 204) open, then the loading mix was added via the sample port in a dropwise fashion. Each sequencing experiment was run on the Oxford Nanopore MinION Mk1B device for 48 hrs, or until all the pores were inactive.

Adaptive sampling was run using the Oxford MinION (v22.03.6) software. A GPU accelerated version of guppy (v6.0.7; API version 10.1.0) was used for basecalling in real-time on two NVIDIA RTX A6000 GPUs using the “super-accurate” model parameters. The adaptive sampling mode was set to deplete the reference human genome (GRCh38.p13) on 256 of the 512 available channels in each run. Reads mapped using Minimap2 (v2.22-r1101).

SMART was used to analyze the output fastq data, and a real-time run simulated by playing back the sequencing run using metadata from the log files (the speed of SMART being more than sufficient to maintain real-time analysis). In brief, this analysis involves selecting a k of 30 for the size of the k-mers, breaking each read into all possible overlapping 30-mers, mapping each 30-mer onto a large database of 30-mers that are unique to species level, then reducing the resulting distribution of candidate matches to the best match at a given taxonomic rank. After each read is mapped to the most likely species, the counts are aggregated and filtered. To obtain the ground truth adenoviral reads for each run, the fastq data was mapped onto a reference genome (KT340071). The read tags were then matched to log data and the SMART assigned species.

We analyzed four samples from the BayNovation adenoviral clinical trial utilizing an example embodiment of the method 400 described above. Characteristics of the samples including qPCR for adenovirus are shown in Table 1; samples ranged from 4.5×10⁵ to 1.3×10⁷ virions/ml. As expected, total reads were higher for adaptive sequencing (as rejected reads are shorter) and ranged from 359,152 to 3,293,797 (Table 2). Total human reads ranged from 95.71% to 96.34% for non-adaptive sequencing, to 84.95% to 85.22% for the adaptive sequencing (Table 3). Fidelity of human sequence was assessed by digital karyotyping. Observed vs. expected reads per chromosome were highly linear suggesting excellent representation of human sequence. All samples were positive for adenoviral sequence, with average number of reads for non-adaptive sequencing ranging from 3 to 9, and for adaptive from 2 to 24 positive reads (Table 4). Post-hoc comparison of SMART-defined positive samples were compared to the MiniMap 2-defined reads, and ranged from 71% recovery to 100% recovery.

We next examined the rapidity with which sequences could be identified (Table 5). For all sequencing, the t ½ for sequencing was 16-24 hours. Time to first real-time identification of adenovirus ranged for non-adaptive sequencing ranged from 2.88 to 19 hours. Time to first real-time identification using adaptive sequencing ranged from 0.5 to 3.74 hours. Adaptive sequencing improved speed-to-first detection by 1.5×-9.4× depending on sample.

Following real-time sequencing, samples were re-analyzed with MiniMap2, which has very high sensitivity for mapping reads to organisms. Average read length for adenoviral sequence ranged from 3.82 to 5.25 KB (Table 6). Average depth of coverage ranged from 0.89 to 4.21. Breadth of coverage ranged from 43% to 75.5%.

As shown above, adaptive sequencing improved speed of detection at least an average of 5× and increased recovery of pathogen sequences by greater than 2-fold. Speed of diagnosis is useful for many infectious diseases. By coupling real-time SMART pipeline function with adaptive sequencing, we were able to detect pathogen DNA with high probability in as little as 30 minutes after start of sequencing run. DNA preparation and library preparation time takes approximately 1 hour, and so diagnostic information would be available in under 2 hours, comparable to the speed of pathogen-directed PCR. This is the fastest pathogen-agnostic detection method described to date, and may yield useful information for patients with unknown diagnoses.

The sensitivity of the technique appears relatively high. The samples chosen for this study had relatively low viral loads; most adenoviral conjunctivitis features viral loads in the 10⁷ range. For the sample (I161118) with highest viral load, time-to-diagnosis was 0.63 hours and total coverage was 4.21× depth with 75.5% breadth at conclusion of the run, allowing both rapid diagnosis and, within 48 hours, substantial genomic characterization of the virus (which may have prognostic information).

TABLE 1
Sample Data

| Sample | AdV Copies | SEI | Treatment |
|---|---|---|---|
| I161756 | 5.90E+06 | POS | NVC-422 |
| S034021 | 5.80E+06 | NEG | NVC-422 |
| I191561 | 4.46E+05 | POS | Vehicle |
| I161118 | 1.30E+07 | NEG | Vehicle |

TABLE 2
Total sequencing reads

| Sample | Adaptive | Non-Adaptive | Total |
|---|---|---|---|
| I191561 | 3293797 | 1413569 | 4707366 |
| I161118 | 359152 | 158504 | 517656 |
| I161756 | 491173 | 313150 | 804323 |
| S034021 | 517958 | 326245 | 844203 |

TABLE 3
Sequencing Results: Human Throughput

| | Counts | | | SMART/Minimap2 Recovery | | | |
|---|---|---|---|---|---|---|---|
| | | Non- | | | Non- | | |
| Sample | Adaptive | Adaptive | Total | Adaptive | Adaptive | Total | Method |
| I191561 | 2502027 | 1267471 | 3769497 | 84.96% | 96.24% | 88.44% | SMART |
| I161118 | 273702 | 141230 | 414932 | 85.40% | 96.29% | 88.82% | SMART |
| I161756 | 379414 | 280063 | 659477 | 85.55% | 95.71% | 89.59% | SMART |
| S034021 | 398481 | 293230 | 691711 | 85.22% | 96.34% | 89.61% | SMART |

TABLE 4
Sequencing Results: HAdV-D8 Throughput

| | Counts | | | SMART/Minimap2 Recovery | | | |
|---|---|---|---|---|---|---|---|
| | | Non- | | | Non- | | |
| Sample | Adaptive | Adaptive | Total | Adaptive | Adaptive | Total | Method |
| I191561 | 5 | 3 | 8 | 71.43% | 50.00% | 61.54% | SMART |
| I161118 | 24 | 6 | 30 | 75.00% | 75.00% | 75.00% | SMART |
| I161756 | 18 | 9 | 27 | 85.71% | 69.23% | 79.41% | SMART |
| S034021 | 2 | 4 | 6 | 100% | 100% | 100% | SMART |

TABLE 5
Adaptive Sampling: Speed to first pathogen read

| | Time | | | Total Reads | |
|---|---|---|---|---|---|
| | | Non- | | | Non- |
| | Adaptive | Adaptive | | Adaptive | Adaptive |
| Sample | [hrs] | [hrs] | Ratio | [N] | [N] |
| I161756 | 0.52 | 4.93 | 9.41 | 3321 | 61040 |
| S034021 | 3.74 | 6.02 | 1.61 | 86041 | 78728 |
| I191561 | 3.38 | 19 | 5.62 | 574114 | 779201 |
| I161118 | 0.63 | 2.88 | 4.53 | 9829 | 13957 |

TABLE 6
Overall coverage following MiniMap2

| | | | Averages | | | | | |
|---|---|---|---|---|---|---|---|---|
| | | | Read length | | | | Breadth | |
| Sample | Reads | Basepairs | [Kbp] | Coverage | BaseQ | MapQ | [Kbp] | [%] |
| I161756 | 34 | 135306 | 3.98 | 3.81 | 22.6 | 60 | 22.9 | 65.65% |
| S034021 | 6 | 31517 | 5.25 | 0.89 | 22 | 60 | 15 | 43.00% |
| I191561 | 13 | 63729 | 4.90 | 1.8 | 22.1 | 60 | 21.4 | 61.22% |
| I161118 | 40 | 152842 | 3.82 | 4.21 | 21.9 | 60 | 26.4 | 75.50% |

As used herein, “phenotype” refers to an appearance of an organism based on a multifactorial combination of genetic traits and environmental factors; a tissue type (e.g., heart tissue vs. adrenal tissue); an organism type (e.g., a strain of bacteria); or an expressed gene.

As used herein, “nanopore” refers to a pore of nanometer size used to generate ionic current changes in response to interactions with molecules present therein.

As used herein, “nucleic acid” refers to a polymer of monomer units or “residues”. The monomer subunits, or residues, of the nucleic acids each contain a nitrogenous base (i.e., nucleobase) a five-carbon sugar, and a phosphate group. The identity of each residue is typically indicated herein with reference to the identity of the nucleobase (or nitrogenous base) structure of each residue. Canonical nucleobases include adenine (A), guanine (G), thymine (T), uracil (U) (in RNA instead of thymine (T) residues) and cytosine (C). However, the nucleic acids of the present disclosure can include any modified nucleobase, nucleobase analogs, and/or non-canonical nucleobase, as are well-known in the art. Modifications to the nucleic acid monomers, or residues, encompass any chemical change in the structure of the nucleic acid monomer, or residue, that results in a noncanonical subunit structure. Such chemical changes can result from, for example, epigenetic modifications (such as to genomic DNA or RNA), or damage resulting from radiation, chemical, or other means. Illustrative and nonlimiting examples of noncanonical subunits, which can result from a modification, include uracil (for DNA), 5-methylcytosine, 5-hydroxymethylcytosine, 5-formethylcytosine, 5-carboxycytosine b-glucosyl-5-hydroxymethylcytosine, 8-oxoguanine, 2-amino-adenosine, 2-amino-deoxyadenosine, 2-thiothymidine, pyrrolo-pyrimidine, 2-thiocytidine, or an abasic lesion. An abasic lesion is a location along the deoxyribose backbone but lacking a base. Known analogs of natural nucleotides hybridize to nucleic acids in a manner similar to naturally occurring nucleotides, such as peptide nucleic acids (PNAs) and phosphorothioate DNA. The five-carbon sugar to which the nucleobases are attached can vary depending on the type of nucleic acid. For example, the sugar is deoxyribose in DNA and is ribose in RNA. In some instances herein, the nucleic acid residues can also be referred with respect to the nucleoside structure, such as adenosine, guanosine, 5-methyluridine, uridine, and cytidine. Moreover, alternative nomenclature for the nucleoside also includes indicating a “ribo” or deoxyrobo” prefix before the nucleobase to infer the type of five-carbon sugar. For example, “ribocytosine” as occasionally used herein is equivalent to a cytidine residue because it indicates the presence of a ribose sugar in the RNA molecule at that residue. A nucleic acid polymer can be or comprise a deoxyribonucleotide (DNA) polymer, a ribonucleotide (RNA) polymer. The nucleic acids can also be or comprise a PNA polymer, or a combination of any of the polymer types described herein (e.g., contain residues with different sugars).

As used herein, “tissue” refers to an aggregate of similar cells and cell products forming a definite kind of structural material with a specific function, in a multicellular organism.

The complete disclosure of all patents, patent applications, and publications, and electronically available material cited herein are incorporated by reference in their entirety. Supplementary materials referenced in publications (such as supplementary tables, supplementary figures, supplementary materials and methods, and/or supplementary experimental data) are likewise incorporated by reference in their entirety. In the event that any inconsistency exists between the disclosure of the present application and the disclosure(s) of any document incorporated herein by reference, the disclosure of the present application shall govern.

The foregoing detailed description and examples have been given for clarity of understanding only. No unnecessary limitations are to be understood therefrom. The disclosure is not limited to the exact details shown and described, for variations obvious to one skilled in the art will be included within the disclosure defined by the claims.

The description of embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure.

Specific elements of any foregoing embodiments can be combined or substituted for elements in other embodiments. Moreover, the inclusion of specific elements in at least some of these embodiments may be optional, wherein further embodiments may include one or more embodiments that specifically exclude one or more of these specific elements. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

As used herein and unless otherwise indicated, the terms “a” and “an” are taken to mean “one”, “at least one” or “one or more”. Unless otherwise required by context, singular terms used herein shall include pluralities and plural terms shall include the singular.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

Unless otherwise indicated, all numbers expressing quantities of components, molecular weights, k-mer lengths and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present disclosure. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

All of the references cited herein are incorporated by reference. Aspects of the disclosure can be modified, if necessary, to employ the systems, functions, and concepts of the above references and application to provide yet further embodiments of the disclosure. These and other changes can be made to the disclosure in light of the detailed description.

It will be appreciated that, although specific embodiments of the disclosure have been described herein for purposes of illustration, various modifications may be made without deviating from the spirit and scope of the disclosure. Accordingly, the disclosure is not limited except as by the claims.

### Examples

Example 1. A computer-implemented method of predicting a molecule type using segment read information, the method comprising: receiving, by a computing device, a segment read for a molecule; determining, by the computing device, one or more k-mers for the segment read; for each k-mer of the one or more k-mers, retrieving, by the computing device, one or more records from a hash table using the k-mer as a key, wherein each record includes an identifier associated with a molecule type; and generating, by the computing device, a molecule type prediction for the segment read based on the identifiers of the retrieved one or more records.

Example 2. The computer-implemented method of example 1, wherein the segment read is based on information generated by a sequencing device; and wherein the method further comprises: in response to determining that the molecule type of at least one retrieved record is an undesired molecule type, transmitting, by the computing device, a command to the sequencing device to abort sequencing of the molecule.

Example 3. The computer-implemented method of any one of example 1 or 2, wherein retrieving the one or more records from the hash table using the k-mer as the key includes using a graphical processing unit (GPU) to retrieve the one or more records from the hash table, wherein the hash table is stored in a memory of the GPU.

Example 4. The computer-implemented method of example 3, wherein the hash table is a WarpCore hash table.

Example 5. The computer-implemented method of any one of examples 1-4, wherein each record from the hash table is associated with an indication of whether the identifier is associated with a unique molecule type, an ambiguous molecule type, or no molecule type.

Example 6. The computer-implemented method of example 5, wherein the method further comprises excluding records from the retrieved one or more records that are not associated with the unique molecule type.

Example 7. The computer-implemented method of any one of examples 1-6, wherein at least one record from the hash table is associated with an indication of a specific feature associated with the k-mer.

Example 8. The computer-implemented method of example 7, wherein the specific feature associated with the k-mer is a chromosomal location.

Example 9. The computer-implemented method of example 7, wherein the specific feature associated with the k-mer is a specific mutation in a genetic locus.

Example 10. The computer-implemented method of any one of examples 1-9, wherein at least one record from the hash table is associated with an indication of a chemical modification of the molecule.

Example 11. The computer-implemented method of example 10, wherein the chemical modification is methylation, acetylation, or another chemical adduct.

Example 12. The computer-implemented method of any one of examples 1-11, wherein receiving the segment read for the molecule includes receiving the segment read for the molecule while a sequencing device is sequencing the molecule.

Example 13. The computer-implemented method of any one of examples 1-12, wherein the molecule is a nucleic acid molecule.

Example 14. The computer-implemented method of example 13, wherein the nucleic acid molecule is DNA or RNA.

Example 15. The computer-implemented method of any one of examples 1-14, wherein the one or more k-mers are determined using a sliding window of fixed length.

Example 16. The computer-implemented method of any one of examples 1-15, wherein generating the molecule type prediction for the segment read includes at least one of generating a cell type prediction, generating a protein family prediction, generating a virus type prediction, generating a tissue type prediction, generating a species prediction, generating an insertion prediction, generating a deletion prediction, generating a point mutation prediction, generating a presence or absence of a gene prediction, or generating a genetic alteration associated with a specific cancer prediction.

Example 17. The computer-implemented method of any one of examples 1-16, wherein generating the molecule type prediction for the segment read based on the identifiers of the retrieved one or more records includes performing an analysis of the identifiers of the retrieved records to determine a most likely identifier based on a distribution of the identifiers.

Example 18. The computer-implemented method of example 17, wherein generating the molecule type prediction for the segment read based on the identifiers of the retrieved one or more records includes retrieving a name of the molecule type using the most likely identifier.

Example 19. A non-transitory computer-readable medium having computer-executable instructions stored thereon that, in response to execution by one or more processors of a computing device, cause the computing device to perform actions of a method as recited in any one of examples 1 to 18.

Example 20. A computing device configured to perform a method as recited in any one of examples 1 to 18.

Example 21. A system for predicting molecule types, the system comprising: a sequencing device; and a computing device communicatively coupled to the sequencing device; wherein the computing device is configured to perform a method as recited in any one of examples 1 to 18.

Example 22. A method of predicting a molecule type in a sample, the method comprising: obtaining the sample from a subject; applying the sample to a sequencing device configured to generate sequence read information; transmitting, by the sequencing device to a computing device, the sequence read information; and performing, by the computing device, actions of a method as recited in any one of examples 1 to 18 to predict the molecule type.

1. A computer-implemented method of predicting a molecule type using segment read information, the method comprising:
receiving, by a computing device, a segment read for a molecule;
determining, by the computing device, one or more k-mers for the segment read;
for each k-mer of the one or more k-mers, retrieving, by the computing device, one or more records from a hash table using the k-mer as a key, wherein each record includes an identifier associated with a molecule type; and
generating, by the computing device, a molecule type prediction for the segment read based on the identifiers of the retrieved one or more records.

2. The computer-implemented method of claim 1, wherein the segment read is based on information generated by a sequencing device; and
wherein the method further comprises:
in response to determining that the molecule type of at least one retrieved record is an undesired molecule type, transmitting, by the computing device, a command to the sequencing device to abort sequencing of the molecule.

3. The computer-implemented method of claim 1, wherein retrieving the one or more records from the hash table using the k-mer as the key includes using a graphical processing unit (GPU) to retrieve the one or more records from the hash table, wherein the hash table is stored in a memory of the GPU.

4. (canceled)

5. The computer-implemented method of claim 1, wherein each record from the hash table is associated with an indication of whether the identifier is associated with a unique molecule type, an ambiguous molecule type, or no molecule type.

6. The computer-implemented method of claim 5, wherein the method further comprises excluding records from the retrieved one or more records that are not associated with the unique molecule type.

7. The computer-implemented method of claim 1, wherein at least one record from the hash table is associated with an indication of a specific feature associated with the k-mer.

8. The computer-implemented method of claim 7, wherein the specific feature associated with the k-mer is a chromosomal location.

9. The computer-implemented method of claim 7, wherein the specific feature associated with the k-mer is a specific mutation in a genetic locus.

10. The computer-implemented method of claim 1, wherein at least one record from the hash table is associated with an indication of a chemical modification of the molecule;
wherein the chemical modification is methylation, acetylation, or another chemical adduct.

11. (canceled)

12. The computer-implemented method of claim 1, wherein receiving the segment read for the molecule includes receiving the segment read for the molecule while a sequencing device is sequencing the molecule.

13. The computer-implemented method of claim 1, wherein the molecule is a nucleic acid molecule.

14. (canceled)

15. The computer-implemented method of claim 1, wherein the one or more k-mers are determined using a sliding window of fixed length.

16. The computer-implemented method of claim 1, wherein generating the molecule type prediction for the segment read includes at least one of generating a cell type prediction, generating a protein family prediction, generating a virus type prediction, generating a tissue type prediction, generating a species prediction, generating an insertion prediction, generating a deletion prediction, generating a point mutation prediction, generating a presence or absence of a gene prediction, or generating a genetic alteration associated with a specific cancer prediction.

17. The computer-implemented method of claim 1, wherein generating the molecule type prediction for the segment read based on the identifiers of the retrieved one or more records includes performing an analysis of the identifiers of the retrieved records to determine a most likely identifier based on a distribution of the identifiers.

18. The computer-implemented method of claim 17, wherein generating the molecule type prediction for the segment read based on the identifiers of the retrieved one or more records includes retrieving a name of the molecule type using the most likely identifier.

19. A non-transitory computer-readable medium having computer-executable instructions stored thereon that, in response to execution by one or more processors of a computing device, cause the computing device to perform actions of a method of predicting a molecule type using segment read information, the actions comprising:
receiving, by a computing device, a segment read for a molecule;
determining, by the computing device, one or more k-mers for the segment read;
for each k-mer of the one or more k-mers, retrieving, by the computing device, one or more records from a hash table using the k-mer as a key, wherein each record includes an identifier associated with a molecule type; and
generating, by the computing device, a molecule type prediction for the segment read based on the identifiers of the retrieved one or more records.

20. (canceled)

21. A system for predicting molecule types, the system comprising:
a sequencing device; and
a computing device communicatively coupled to the sequencing device;
wherein the computing device includes at least one processor and a non-transitory computer readable medium having computer-executable instructions stored thereon that, in response to execution by the at least one processor, cause the system to perform actions of a method of predicting a molecule type using segment read information, the actions comprising:
receiving, by the computing device from the sequencing device, a segment read for a molecule;
determining, by the computing device, one or more k-mers for the segment read;
for each k-mer of the one or more k-mers, retrieving, by the computing device, one or more records from a hash table using the k-mer as a key, wherein each record includes an identifier associated with a molecule type; and
generating, by the computing device, a molecule type prediction for the segment read based on the identifiers of the retrieved one or more records.

22. (canceled)

23. The system of claim 21, wherein the segment read is based on information generated by a sequencing device; and
wherein the actions further comprise:
in response to determining that the molecule type of at least one retrieved record is an undesired molecule type, transmitting, by the computing device to the sequencing device, a command to abort sequencing of the molecule.

24. The system of claim 21, wherein the computing device further includes a graphical processing unit (GPU);
wherein the hash table is stored in a memory of the GPU; and
wherein retrieving the one or more records from the hash table using the k-mer as the key includes using, by the computing device, the GPU to retrieve the one or more records from the hash table in the memory of the GPU.

25. The system of claim 21, wherein each record from the hash table is associated with an indication of whether the identifier is associated with a unique molecule type, an ambiguous molecule type, or no molecule type; and
wherein the actions further comprise excluding records from the retrieved one or more records that are not associated with the unique molecule type.
